(19) United States
(12) Patent Application Publication
(10) Pub. No.: US 2013/0150315 A1
(43) Pub. Date: Jun. 13, 2013

(54) Anti-Protozoal for Apicomplexan Protozoa

(71) Applicant: Mary C. Kline, Glen Rock, PA (US)

(72) Inventor: Mary C. Kline, Glen Rock, PA (US)

(21) Appl. No.: 13/708,982

(22) Filed: Dec. 8, 2012

Related U.S. Application Data
(60) Provisional application No. 61/568,743, filed on Dec. 9, 2011.

(51) Int. Cl. A61K 31/47; A61K 31/7048; A61K 31/7052; A61K 31/4164; A61K 31/357

(52) U.S. Cl. CPC A61K 31/47; A61K 31/4164; A61K 31/357; A61K 31/7052; A61K 31/7048
USPC 514/29; 514/312

(57) ABSTRACT

The invention comprising the heretofore veterinary anti-protozoal decoquinate and one or both of a redox drug or macrolide drug for prophylaxis or treatment of Babesia and other Apicomplexan protozoa infections in humans. The decoquinate or its metabolite kill certain life stages of Babesia. The redox and macrolide drugs treat life stages and forms of Babesia that are not susceptible to the decoquinate.

### CROSS REFERENCE TO RELATED APPLICATIONS

Provisional application 61/568,743

### BACKGROUND OF THE INVENTION

1. Field of the Invention

The invention pertains to human drugs. More specifically the invention pertains to a drug combination for the treatment and prevention of Apicomplexan protozoal infections in humans.

2. Description of Related Art

Babesiosis is a protozoa disease of wild and domestic animals, and humans. Babesiosis in humans is generally associated with B. divergens, B. duncanii, and B. macroti. Tests for antibodies to the protozoa antigens are incomplete and not sensitive. Specialists acknowledge the lack of a good test for the disease, and have suggested ‘Babesia-like organisms’ as the cause of some cases.

Babesiosis is a serious disease causing malaria-like symptoms of fatigue, anemia, and neurologic symptoms. The most common route of infection is through the bite of an infected tick. Other vectors of infection have been suggested by researchers. The incidence of Babesiosis varies by geographic location, and reports of the disease are rising. Babesia infects the red blood cells and can be disseminated through the tissues, and planktonic or sequestered in the vasculature. Babesiosis is now a reportable disease in the United States. Once infected with Babesia, a human should never donate blood or tissues. Babesia can be acquired through blood transfusions and transplants. Transfusion and transplant patients are not currently given prophylactic anti-protozoal drugs due to both cost and possible serious side effects.

Protomyxzoa is an emerging Apicomplexan protozoa disease of animals and humans. This protozoon sequesters protozoa and other pathogens in the vasculature in biofilms or plaques. Active cells and persister cells of various protozoa, bacteria, viruses, and fungi are encapsulated in the biofilms making anti-microbial treatment less effective or ineffective on the cells. No current drug utilizes reduction of these polymicrobial biofilms as a pathway or target to treat the persister cells.

Babesia and other Apicomplexan protozoa have multiple life stages and forms. Some stages may be present only in the peripheral blood stream, other stages may be sequestered in vasculature biofilms or plaques, or encysted in tissue. Some Apicomplexan protozoa infect white blood cells, and others infect red blood cells. No single drug has been found efficacious on all life stages or forms of Apicomplexan protozoa. Multiple mechanisms of drug action are necessary to eliminate the pathogen in multiple stages and forms. No drug has previously been identified for prevention or treatment of Babesia or Protomyxzoa sequestered in biofilms or plaques.

Babesia and other Apicomplexan protozoa must complete at least one life stage inside of white or red blood cells. Current treatment protocol of Plasmodium and Babesia involve Atovaquone alone or in combination with other drugs. Additional drug combinations have been suggested with other FDA-approved human anti-protozoal drugs. All of these drugs have larger, more complex molecules, which limit their ability to enter cells and the central nervous system, and are not known to reduce biofilms and plaques. The current Babesiosis drug protocol of atovaquone can have serious side effects and is costly. Atovaquone treatment is suggested for five months, which may not be possible due to cost and side effects. Babesiosis and other apicomplexan protozoa are showing resistance to Atovaquone. A more efficacious, safer, and faster drug protocol is needed.

### BRIEF SUMMARY OF THE INVENTION

The invention comprising the heretofore veterinary anti-protozoal decoquinate and one or both of a redox drug or macrolide drug for prophylaxis or treatment of Babesia infections in humans. The decoquinate or its metabolite kill certain life stages of Babesia. The redox and macrolide drugs treat life stages and forms of Babesia that are not susceptible to the decoquinate. Decoquinate or its metabolites reduce biofilms which sequester active and persister cells of various pathogens making the pathogens more susceptible to the redox and/or macrolide drugs. Decoquinate has not been specified before to reduce polymicrobial biofilms. The decoquinate is more efficacious than other heretofore human anti-protozoals by its small molecule size enabling it to better enter cells, being faster acting, and it has no known side effects.

### DETAILED DESCRIPTION OF THE INVENTION

The invention comprising decoquinate: 3-Quinolinecarboxylic acid,6-(decyloxy)-7-ethoxy-4-hydroxy-,ethyl ester; Ethyl 6-(decyloxy)-7-ethoxy-4-hydroxy-3-quinolinecarboxylate [18507-89-6]; and its metabolites with one or both of a redox drug and a macrolide drug, or pharmaceutically acceptable salts thereof. Pharmaceutical excipients may be added as necessary to obtain the dosage or form.

The redox drug is such as metronidazole: 2-(2-methyl-5-nitroimidazol-1-yl)ethanol; or artemisinin or a derivative of artemisinin or pharmaceutically acceptable salts thereof.

The macrolide drug is such as azithromycin: 2R,3S,4R,5R,8R,10R,11R,12S,13S,14R)-13-[(2,6-dideoxy-3-C-methyl-3-O-methyl-a-L-ribo-hexopyranosyl)oxy]-2-ethyl-3,4,10-trihydroxy-3,5,6,8,10,12,14-heptamethyl-11-[[3,4,6-trideoxy-3-(dimethylamino)-b-D-xylo-hexopyranosyl]oxy]-1-oxa-6-azacyclopentadecan-15-one; or clarithromycin: (3R,4S,5S,6R,7R,9R,11R,12R,13S,14R)-6-[(2S,3R,4S,6R)-4-(dimethylamino)-3-hydroxy-6-methyloxan-2-yl]oxy-14-ethyl-12,13-dihydroxy-4-[(2R,4R,5S,6S)-5-hydroxy-4-methoxy-4,6-dimethyloxan-2-yl]oxy-7-methoxy-3,5,7,9,11,13-hexamethyl-oxacyclotetradecane-2,10-dione or pharmaceutically acceptable salts thereof.

Decoquinate kills certain life stages of Babesia and other Apicomplexan protozoa. Decoquinate or its metabolites also reduce the matrix and pathogen population of polymicrobial biofilms and plaques of the vasculature and tissues. The return of biofilm pathogens to a planktonic state allows better efficacy or of the redox or macrolide drugs. The reduction of biofilms exposes sequestered persister cells or induces them to become active, allowing better efficacy of the redox or macrolide drugs. Decoquinate has not been used before to reduce biofilms or plaques.

Prophylactic treatment during transfusions or transplants could aid those patients by prohibiting infection by Babesia without drug side effects. Prophylactic treatment would be useful during travel to areas where certain Apicomplexan protozoa are endemic. Several Apicomplexan protozoa infect humans, and this drug combination would be efficacious against Toxoplasma, Coccidea, Protomyxzoa, Plasmodium, and other Apicomplexan protozoa.

In treating apicomplexan protozoal infections, patients with high parasitemia rates may experience effects from toxins released from the killed pathogens. It may be beneficial to patients to clear different life stages of the pathogen sequentially, lessening the load of toxin released simultaneously. The invention also includes the order of combination of the drugs, starting the decoquinate first, to clear levels of parasitemia from the blood stream and to reduce the biofilms. The redox drug and or macrolide can be added to the decoquinate later. The drugs should be combined, as opposed to given singly in sequence, to keep the pathogen from changing to forms not killed by the single drug given.

The invention is for oral administration in various forms such as tablet, gelatin capsule, capsule, suspensions, paste or lozenges; and for intravenous administration; and for use cutaneously or on external bandages. Decoquinate has a very high therapeutic index in domestic animals. The dosage for humans is weight and age dependant, and will differ dependant on use as prophylaxis or treatment. Protozoa infecting the red blood cells will require a higher dosage than protozoa infecting the white blood cells. The decoquinate dosage is 0.1 mg/kg to 40.0 mg/kg daily. The dosage of the two redox drug examples are metronidazole at 50 mg to 3000 mg daily, or artemisinin at 50 mg to 3000 mg daily. The dosage for the two macrolide drug examples are azithromycin or clarithromycin at 50 to 3000 mg daily.

1. A drug for prophylaxis or treatment of babesia in humans, including babesia sequestered in polymicrobial biofilms and plaque, comprising decoquinate: ethyl 6-decoxy-7-ethoxy-4-oxo-1H-quinoline-3-carboxylate [18507-89-6]; and a redox-active drug, or pharmaceutically acceptable salts thereof.

2. The drug in claim 1 wherein the redox-active drug is metronidazole: 2-(2-methyl-5-nitroimidazol-1-yl)ethanol; or pharmaceutically acceptable salts thereof.

3. The drug in claim 1 wherein the redox-active drug is artemisinin or a derivative of artemisinin, or pharmaceutically acceptable salts thereof.

4. A drug for prophylaxis or treatment of babesia in human, including babesia sequestered in polymicrobial biofilms and plaque, comprising the drug in claim 1 and a macrolide drug, or pharmaceutically acceptable salts thereof.

5. The drug in claim 4 wherein the macrolide drug is azithromycin: (2R,3S,4R,5R,8R,10R,11R,12S,13S,14R)-11-[(2S,3R,4S,6R)-4-(dimethylamino)-3-hydroxy-6-methyloxan-2-yl]oxy-2-ethyl-3,4,10-trihydroxy-13-[(2R,4R,5S,6S)-5-hydroxy-4-methoxy-4,6-dimethyloxan-2-yl]oxy-3,5,6,8,10,12,14-heptamethyl-1-oxa-6-azacyclopentadecan-15-one; or pharmaceutically acceptable salts thereof.

6. The drug in claim 4 wherein the macrolide drug is clarithromycin: (3R,4S,5S,6R,7R,9R,11R,12R,13S,14R)-6-[(2S,3R,4S,6R)-4-(dimethylamino)-3-hydroxy-6-methyloxan-2-yl]oxy-14-ethyl-12,13-dihydroxy-4-[(2R,4R,5S,6S)-5-hydroxy-4-methoxy-4,6-dimethyloxan-2-yl]oxy-7-methoxy-3,5,7,9,11,13-hexamethyl-oxacyclotetradecane-2,10-dione; or pharmaceutically acceptable salts thereof.

7. The drug in claim 1 for prophylaxis or treatment of protomyxzoa in humans, including protomyxzoa sequestered in polymicrobial biofilms and plaques.

8. The drug in claim 1 for prophylaxis or treatment of toxoplasma in humans, including toxoplasma sequestered in polymicrobial biofilms and plaque.

9. The drug in claim 1 for prophylaxis or treatment of coccidea in humans, including coccidea sequestered in polymicrobial biofilms or plaques.

10. The drug in claim 1 for prophylaxis or treatment of plasmodium in humans, including plasmodium sequestered in polymicrobial biofilms or plaques.

11. The drug in claim 1 for prophylaxis or treatment of other apicomplexan protozoa infections in humans, including in polymicrobial biofilms and plaques in humans.

12. The drug in claim 1 for treatment or reduction of polymicrobial biofilms and plaques in humans.

13. The drug in claim 4 for prophylaxis or treatment of protomyxzoa in humans, including protomyxzoa sequestered in polymicrobial biofilms and plaques.

14. The drug in claim 4 for prophylaxis or treatment of toxoplasma in humans, including toxoplasma sequestered in polymicrobial biofilms and plaque.

15. The drug in claim 4 for prophylaxis or treatment of plasmodium in humans, including plasmodium sequestered in polymicrobial biofilms or plaques.

16. The drug in claim 4 for treatment or reduction of polymicrobial biofilms and plaques in humans.
